(19) United States
(12) Patent Application Publication
(10) Pub. No.: US 2026/0209708 A1
(43) Pub. Date: Jul. 23, 2026

(54) MOLECULAR NANOPARTICLE MICROCARRIERS AND RELATED METHODS

(71) Applicant: NANJING DRUM TOWER HOSPITAL, Nanjing (CN)

(72) Inventors: Lingyun SUN, Nanjing (CN); Min NIE, Nanjing (CN); Yuanjin ZHAO, Nanjing (CN)

(73) Assignee: NANJING DRUM TOWER HOSPITAL, Nanjing (CN)

(21) Appl. No.: 19/572,845

(22) Filed: Mar. 19, 2026

Related U.S. Application Data
(63) Continuation of application No. PCT/CN2024/123515, filed on Oct. 9, 2024.

(30) Foreign Application Priority Data
Oct. 13, 2023 (CN) 202311326390.7

(51) Int. Cl. C12N 5/0775; A61K 35/28; C07K 14/705; C07K 14/715; C12N 15/113; C12N 15/86; C12N 15/88

(52) U.S. Cl. CPC C12N 5/0662; A61K 35/28; C07K 14/70596; C07K 14/7158; C12N 15/1136; C12N 15/1138; C12N 15/86; C12N 15/88; C12N 2310/14; C12N 2740/15043; C12N 2740/15052

(57) ABSTRACT

Provided is molecular nanoparticle microcarriers and the related methods. The preparation method of the molecular nanoparticle microcarriers includes: S1, stimulating mesenchymal stem cells (MSCs) to produce vesicles and collecting the vesicles by centrifugation; wherein the MSCs overexpress CXCR4 and PSGL-1, and a preparation process of the MSCs includes: (a) constructing a recombinant vector of CXCR4 and PSGL-1 using molecular biology techniques; (b) packaging lentivirus in 293T cells using the recombinant vector of CXCR4 and PSGL-1; and (c) collecting the lentivirus, infecting MSCs with concentrated lentivirus, and screening positive cells expressing both CXCR4 and PSGL-1 using antibiotic tags and a flow cytometry to obtain a stable MSCs line that overexpresses CXCR4 and PSGL-1; S2, preparing small interfering ribonucleic acids-loaded polypeptide dendrimer nanoparticles (PDNPs-siRNAs); and S3, mixing and extruding the vesicles with the PDNPs-siRNAs, to obtain the molecular nanoparticle microcarriers (M@PDNPs-siRNAs).

### CROSS-REFERENCE TO RELATED APPLICATIONS

This application is a continuation of International Application No. PCT/CN2024/123515, filed on Oct. 9, 2024, which claims priority of Chinese Patent Application No. 202311326390.7, filed on Oct. 13, 2023, the entire contents of each of which are incorporated herein by reference.

### SEQUENCE LISTING

The instant application contains a Sequence Listing which has been submitted electronically in XML format and is hereby incorporated by reference in its entirety. The XML copy, created on Mar. 18, 2026, is named “2026 Mar. 18-Sequence listing-68401-H015US00” and is 8,766 bytes in size.

### TECHNICAL FIELD

The present disclosure generally relates to the technical field of biomaterials, and in particular, to a method for preparing a molecular nanoparticle microcarriers and related methods.

### BACKGROUND

Rheumatoid arthritis (RA) is a chronic systemic autoimmune disease with complex pathophysiological mechanisms, characterized by synovial lining hyperplasia, angiogenesis, pannus formation, and destruction of cartilage and bone. Clinically, non-steroidal anti-inflammatory drugs (NSAIDs), disease-modifying anti-rheumatic drugs (ADRs), and glucocorticoids have been utilized to ameliorate RA symptoms. Considering drug resistance and adverse effects caused by prolonged and high-dose administration, more effective and targeted therapeutic methods have been developed. Monoclonal antibody preparations targeting pro-inflammatory cytokines, such as infliximab and tocilizumab, have been demonstrated effective for patients with RA. However, monoclonal antibody therapy is not as efficacious as conventional medications due to the limited retention capacity of the monoclonal antibodies in target tissues, its low bioavailability, and adverse effects associated with systemic administration. Compared to biological agents, small interfering RNAs (siRNAs) have inherent advantages such as interfering with mRNA translation and regulating downstream protein expression. However, the suboptimal stability of siRNAs has limited their widespread clinical application.

To address this problem, the present disclosure provides a novel siRNA-targeted delivery system, which consists of peptide dendrimer nanoparticles (PDNPs-siRNAs) loaded with siRNAs carried by cell membranes of mesenchymal stem cells (MSCs), for the treatment of rheumatoid arthritis by intravenous administration. Compared with traditional linear polymers, polypeptide dendrimers possess a highly branched structure, protein-like globular structure, and a large quantity of peripheral groups, making them ideal carriers. Encapsulation of siRNAs into peptide dendrimer nanoparticles (PDNPs) by electrostatic adsorption increases the stability of siRNAs and enables sustained release of therapeutic siRNAs. In addition, bioengineering techniques are introduced to endow MSCs with chemotactic receptors and endothelial cell adhesion molecules, which mediate homing to inflamed and lesioned tissues. P-selectin glycoprotein ligand-1 (PSGL-1) is a glycoprotein expressed on the surface of all leukocytes that specifically targets the vascular system of damaged knee synovial tissues expressing high levels of the PSGL-1 ligands, namely E-selectin and P-selectin. The prepared microcarriers, in the presence of biological signals, can cross the vascular barrier and reach lesioned tissues. C-X-C chemokine receptor 4 (CXCR4) is a G protein-coupled transmembrane receptor. Its ligand, stromal cell-derived factor 1a (SDF-1a), is highly expressed on synoviocytes and a major regulator of synovial chemotaxis. Thus, the present disclosure integrates MSCs-derived cell membranes with high expression of PSGL-1 and CXCR4, PDNPs, IL-6 siRNA, and HIF1-α siRNA, which can be utilized as specific targeted delivery system for RA therapy.

### SUMMARY

The purpose of the present disclosure is to provide, in response to the deficiencies of the prior art, a preparation method of molecular nanoparticle microcarriers and related methods, which solves the problems of the traditional drugs with numerous side effects, low drug absorption rate, and lack of targeting.

In order to achieve the above purpose, the present disclosure provides a preparation method of molecular nanoparticle microcarriers, including: S1, stimulating mesenchymal stem cells (MSCs) to produce vesicles and collecting the vesicles by centrifugation; wherein the MSCs overexpress CXCR4 and PSGL-1, and a preparation process of the MSCs includes: (a) constructing a recombinant vector of CXCR4 and PSGL-1 using molecular biology techniques; (b) packaging lentivirus in 293T cells using the recombinant vector of CXCR4 and PSGL-1; and (c) collecting the lentivirus, infecting MSCs with concentrated lentivirus, and screening positive cells expressing both CXCR4 and PSGL-1 using antibiotic tags and a flow cytometry to obtain a stable MSCs line that overexpresses CXCR4 and PSGL-1; S2, preparing small interfering ribonucleic acids-loaded polypeptide dendrimer nanoparticles (PDNPs-siRNAs); and S3, mixing and extruding the vesicles with the PDNPs-siRNAs, to obtain the molecular nanoparticle microcarriers (M@PDNPs-siRNAs), wherein human CXCR4 sequence is set forth in SEQ ID NO: 1 and human PSGL-1 sequence is set forth in SEQ ID NO: 2.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, wherein:

FIG. 1 is a schematic diagram of MSCs vesicles overexpressing CXCR4 and PSGL-1 according to Example 1 of the present disclosure;

FIG. 2 is an agarose gel electrophoresis (AGE) image of small interfering ribonucleic acids-loaded polypeptide dendrimer nanoparticles (PDNPs-siRNAs) according to Example 2 of the present disclosure;

FIG. 3 is a transmission electron microscope (TEM) image of a molecular nanoparticle microcarrier (M@PDNPs-siRNAs) according to Example 3 of the present disclosure;

FIG. 4 is a chart showing the effect of inhibiting target gene expression after transfection of synovial fibroblasts with M@PDNPs-siRNAs according to some embodiments of the present disclosure;

FIG. 5 is an image showing the effect of targeting validation of M@PDNPs-siRNAs according to some embodiments of the present disclosure.

### DETAILED DESCRIPTION

In the following detailed description, numerous specific details are set forth by way of examples in order to provide a thorough understanding of the relevant disclosure. Obviously, drawings described below are only some examples or embodiments of the present disclosure. Those skilled in the art, without further creative efforts, may apply the present disclosure to other similar scenarios according to these drawings. It should be understood that the purposes of these illustrated embodiments are only provided to those skilled in the art to practice the application, and not intended to limit the scope of the present disclosure. Unless obviously obtained from the context or the context illustrates otherwise, the same numeral in the drawings refers to the same structure or operation.

In some embodiments, a preparation method of a molecular nanoparticle microcarrier includes operations S1 to S3:

S1, stimulating mesenchymal stem cells (MSCs) to produce vesicles and collecting the vesicles by centrifugation.

MSCs are adult stem cells with multidirectional differentiation potential and immunomodulatory functions, which are widely present in bone marrow, adipose, umbilical cord, and other tissues.

In some embodiments, in S1, the MSCs are MSCs overexpressing C-X-C chemokine receptor 4 (CXCR4) and P-selectin glycoprotein ligand-1 (PSGL-1), and the preparation process of the MSCs includes operations (a) to (c):

(a) constructing a recombinant vector of CXCR4 and PSGL-1 using molecular biology techniques.

CXCR4 is a G-protein-coupled transmembrane receptor, and its ligand is stromal cell-derived factor 1a (SDF-1a). In RA lesions, synoviocytes highly express SDF-1a. CXCR4, as its specific receptor, mediates the chemotactic migration of cells toward the inflammatory sites with a high SDF-1a concentration gradient (e.g., lesioned synovial tissues), and it is a major regulator of synovial chemotaxis.

PSGL-1 is a glycoprotein expressed on the surface of all leukocytes, and its ligands are E-selectin and P-selectin. In RA lesions, the vascular system of knee synovial tissues highly expresses the E-selectin and the P-selectin as a result of inflammatory stimulation. The PSGL-1 mediates adhesion of cells to the vascular endothelium at the inflammatory sites by interacting with these ligands, which in turn allows cells to penetrate the vascular barrier to reach the lesioned tissues.

In some embodiments, human CXCR4 sequence is
(SEQ ID NO: 1)
ATGTCCATTCCTTTGCCTCTTTTGCAGATATACACTTCAGATAAC
TACACCGAGGAAATGGGCTCAGGGGACTATGACTCCATGAAGGAA
CCCTGTTTCCGTGAAGAAAATGCTAATTTCAATAAAATCTTCCTG
CCCACCATCTACTCCATCATCTTCTTAACTGGCATTGTGGGCAAT
GGATTGGTCATCCTGGTCATGGGTTACCAGAAGAAACTGAGAAGC
ATGACGGACAAGTACAGGCTGCACCTGTCAGTGGCCGACCTCCTC
TTTGTCATCACGCTTCCCTTCTGGGCAGTTGATGCCGTGGCAAAC
TGGTACTTTGGGAACTTCCTATGCAAGGCAGTCCATGTCATCTAC
ACAGTCAACCTCTACAGCAGTGTCCTCATCCTGGCCTTCATCAGT
CTGGACCGCTACCTGGCCATCGTCCACGCCACCAACAGTCAGAGG
CCAAGGAAGCTGTTGGCTGAAAAGGTGGTCTATGTTGGCGTCTGG
ATCCCTGCCCTCCTGCTGACTATTCCCGACTTCATCTTTGCCAAC
GTCAGTGAGGCAGATGACAGATATATCTGTGACCGCTTCTACCCC
AATGACTTGTGGGTGGTTGTGTTCCAGTTTCAGCACATCATGGTT
GGCCTTATCCTGCCTGGTATTGTCATCCTGTCCTGCTATTGCATT
ATCATCTCCAAGCTGTCACACTCCAAGGGCCACCAGAAGCGCAAG
GCCCTCAAGACCACAGTCATCCTCATCCTGGCTTTCTTCGCCTGT
TGGCTGCCTTACTACATTGGGATCAGCATCGACTCCTTCATCCTC
CTGGAAATCATCAAGCAAGGGTGTGAGTTTGAGAACACTGTGCAC
AAGTGGATTTCCATCACCGAGGCCCTAGCTTTCTTCCACTGTTGT
CTGAACCCCATCCTCTATGCTTTCCTTGGAGCCAAATTTAAAACC
TCTGCCCAGCACGCACTCACCTCTGTGAGCAGAGGGTCCAGCCTC
AAGATCCTCTCCAAAGGAAAGCGAGGTGGACATTCATCTGTTTCC
ACTGAGTCTGAGTCTTCAAGTTTTCACTCCAGCTAA,
and human PSGL-1 sequence is
(SEQ ID NO: 2)
ATGGCAGTGGGGGCCAGTGGTCTAGAAGGAGATAAGATGGCTGGT
GCCATGCCTCTGCAACTCCTCCTGTTGCTGATCCTACTGGGCCCT
GGCAACAGCTTGCAGCTGTGGGACACCTGGGCAGATGAAGCCGAG
AAAGCCTTGGGTCCCCTGCTTGCCCGGGACCGGAGACAGGCCACC
GAATATGAGTACCTAGATTATGATTTCCTGCCAGAAACGGAGCCT
CCAGAAATGCTGAGGAACAGCACTGACACCACTCCTCTGACTGGG
CCTGGAACCCCTGAGTCTACCACTGTGGAGCCTGCTGCAAGGCGT
TCTACTGGCCTGGATGCAGGAGGGGCAGTCACAGAGCTGACCACG
GAGCTGGCCAACATGGGGAACCTGTCCACGGATTCAGCAGCTATG
GAGATACAGACCACTCAACCAGCAGCCACGGAGGCACAGACCACT
CAACCAGTGCCCACGGAGGCACAGACCACTCCACTGGCAGCCACA
GAGGCACAGACAACTCGACTGACGGCCACGGAGGCACAGACCACT
CCACTGGCAGCCACAGAGGCACAGACCACTCCACCAGCAGCCACG
GAAGCACAGACCACTCAACCCACAGGCCTGGAGGCACAGACCACT
GCACCAGCAGCCATGGAGGCACAGACCACTGCACCAGCAGCCATG
GAAGCACAGACCACTCCACCAGCAGCCATGGAGGCACAGACCACT
CAAACCACAGCCATGGAGGCACAGACCACTGCACCAGAAGCCACG
GAGGCACAGACCACTCAACCCACAGCCACGGAGGCACAGACCACT
CCACTGGCAGCCATGGAGGCCCTGTCCACAGAACCCAGTGCCACA
GAGGCCCTGTCCATGGAACCTACTACCAAAAGAGGTCTGTTCATA
CCCTTTTCTGTGTCCTCTGTTACTCACAAGGGCATTCCCATGGCA
GCCAGCAATTTGTCCGTCAACTACCCAGTGGGGGCCCCAGACCAC
ATCTCTGTGAAGCAGTGCCTGCTGGCCATCCTAATCTTGGCGCTG
GTGGCCACTATCTTCTTCGTGTGCACTGTGGTGCTGGCGGTCCGC
CTCTCCCGCAAGGGCCACATGTACCCCGTGCGTAATTACTCCCCC
ACCGAGATGGTCTGCATCTCATCCCTGTTGCCTGATGGGGGTGAG
GGGCCCTCTGCCACAGCCAATGGGGGCCTGTCCAAGGCCAAGAGC
CCGGGCCTGACGCCAGAGCCCAGGGAGGACCGTGAGGGGGATGAC
CTCACCCTGCACAGCTTCCTCCCTTAG.

The recombinant vector are plasmids containing target sequences such as CXCR4 and PSGL-1. The recombinant vector may be integrated into host cells by lentivirus, to efficiently express the exogenous gene in the host cells.

In some embodiments, the recombinant vector may also contain a FLAG tag, a fluorescent protein sequence (e.g., an EGFP sequence), an antibiotic resistance sequence (e.g., a puromycin resistance sequence), or the like. The FLAG tag has selective adsorption, which facilitates the purification of recombinant vectors; and the fluorescence ability brought by the fluorescent protein sequence and antibiotic resistance ability brought by the antibiotic resistance sequence, facilitates the determination of whether the host cells are successfully infected.

In some embodiments, the recombinant vector may be plv-IRES, the plv-IRES-CXCR4 and plv-IRES-PSGL-1 may be obtained by inserting the target sequence (CXCR4 or PSGL-1), a plurality of repetitive FLAG tag sequences, the EGFP sequence, and the puromycin resistance sequence into the open reading frame of plv-IRES.

In some embodiments, the molecular biology techniques include gene synthesis, PCR amplification, or the like.

(b) packaging lentivirus in 293T cells using the recombinant vector of CXCR4 and PSGL-1.

The 293T cells are immortalized engineered tool cells, commonly used as a virus packaging tool cells in genetic engineering.

The lentivirus is a genus of retroviruses, which is commonly used as gene delivery tools in genetic engineering.

In some embodiments, a lentiviral packaging system is employed, to package lentivirus in 293T cells. The lentiviral packaging system may be determined based on practical needs. For example, the lentiviral packaging system includes plv-IRES-CXCR4 and plv-IRES-PSGL-1, psPAX2, PMD2G.

In some embodiments, the lentiviral packaging system, a co-transfection reagent (e.g., p3000), and a transfection reagent (e.g., Lip3000) are mixed, and the mixture is added dropwise into well-cultured 293T cell suspension, after cultivation, the genes of the lentivirus are transcribed into the 293T cells, allowing for the expansion of large quantities of lentivirus by the 293T cells. The supernatant is collected to obtain the lentiviral suspension.

(c) collecting the lentivirus, infecting MSCs with concentrated lentivirus, and screening positive cells expressing both CXCR4 and PSGL-1 using antibiotic tags and flow cytometry to obtain a stable MSCs line that overexpresses CXCR4 and PSGL-1.

In some embodiments, the collected lentiviral suspension and the co-transfection reagent may be mixed with the well-cultivated MSCs, after cultivation and screening, the MSCs overexpressing CXCR4 and PSGL-1 are obtained.

The screening manner may include an antibiotic screening manner and a fluorescence screening manner. Specific antibiotics are added to the cultured MSCs. Since the unsuccessfully transfected MSCs do not contain the antibiotic resistance sequence, the unsuccessfully transfected MSCs is unable to resist to the specific antibiotics and thus will die. The surviving MSCs are subjected to fluorescence screening again. Since the successfully transfected MSCs carry the fluorescent protein sequence and may produce fluorescent protein, the MSCs with fluorescent protein may be screened out by flow cytometry. After the dual screening, the retained MSCs may be regarded as the successfully transfected MSCs, and the MSCs overexpressing CXCR4 and PSGL-1 are obtained.

The vesicles are membrane vesicle structures shed from the surface of the MSCs. The MSCs overexpressing CXCR4 and PSGL-1 (hereafter referred to as overexpressing MSCs) may secrete vesicles carrying CXCR4 and PSGL-1.

In some embodiments, the overexpressing MSCs may be stimulated in a plurality of ways. For example, a chemical induction manner may be used to stimulate the release of vesicles by adding compounds or biomolecules. As another example, a physical stimulation manner may be used to stimulate the release of vesicles by low temperature, cyclic variable temperature, hypoxic environment, anoxic environments, or the like. For example, the compounds or biomolecules include prostaglandin E2, calcium ion carriers, lipopolysaccharides, etc. The vesicles may be obtained by centrifugal collection after stimulation of the overexpressing MSCs.

In some embodiments, in S1, the preparation process of the vesicles includes: treating the MSCs overexpressing CXCR4 and PSGL-1 with cytochalasin B, and obtaining the vesicles of the MSCs overexpressing CXCR4 and PSGL-1 by ultra-high speed centrifugation.

The cytochalasin B is a small molecule compound extracted from a fungus, which belongs to a class of actin polymerization inhibitors. It specifically disrupts microfilament structures in the cytoskeleton, induces changes in the morphology of cell membranes, and promotes vesicle release. The added amount and the treatment time of cytochalasin B may be determined according to the actual application scenarios and needs.

In some embodiments, the working concentration of cytochalasin B is 10 μg/mL, and the treatment time is 30 minutes.

In some embodiments, the centrifugation speed and centrifugation temperature of the ultra-high speed centrifugation manner may be determined based on actual application scenarios and needs.

In some embodiments, the operations of the ultra-high speed centrifugation manner include: digesting the treated cells with trypsin, centrifuging at 1000 rpm at room temperature for 5 minutes, washing once with phosphate buffered saline (PBS), and centrifuging again at 1000 rpm at room temperature for 5 minutes; taking the supernatant, centrifuging the supernatant at 6000 rpm at room temperature for 15 minutes, and obtaining precipitate, which is the vesicles.

In some embodiments, since the overexpressing MSCs are cultured in an adherent manner, the overexpressing MSCs are usually tightly adhered to the surface of the culture vessel, and cell clusters also form between cells. By using the trypsin, the cells may be detached from the culture vessel and dispersed into individual cells. Compared to manners such as mechanical scraping, using the trypsin can reduce damage to cells and vesicle structure. By washing with PBS, rupture of cells or vesicles due to sudden changes in osmotic pressure may be avoided during washing.

In some embodiments, after vesicles are obtained, the quality of the vesicles may be assessed by determining the protein concentrations of CXCR4 and PSGL-1 on the vesicles. The vesicles are considered non-qualified when the protein concentration is less than a preset value.

In some embodiments, by treating the overexpressing MSCs with cytochalasin B and combining with ultra-high speed centrifugation to isolate vesicles, this operation can efficiently induce the vesicle release, while safeguarding the structural integrity of the vesicles, avoiding the vesicle rupture, and ensuring the activity of CXCR4 and PSGL-1 receptors on the vesicle surface.

S2, preparing small interfering ribonucleic acids-loaded polypeptide dendrimer nanoparticles (PDNPs-siRNAs).

The small interfering RNAs (siRNAs) are a class of double-stranded RNA molecules, about 20-25 nucleotides in length, that may specifically recognize and degrade target gene mRNAs, thereby inhibiting gene expression and achieving post-transcriptional gene silencing. By choosing different types of siRNAs, the PDNPs-siRNAs may be applied to different diseases. For example, the siRNAs include IL-6 interfering siRNA and HIF-1α interfering siRNA. The IL-6 interfering siRNA and HIF-1a interfering siRNA may be obtained in a variety of ways, e.g., by self-synthesis or commercial purchase.

In some embodiments, the IL-6 interfering siRNA includes a sense strand and an antisense strand:

The sense strand:
(SEQ ID NO: 3)
5′-CCUCUGGUCU UCUGGAGUATT-3′.
The antisense strand:
(SEQ ID NO: 4)
5′-UACUCCAGAAGACCAGAGGTT-3′.

The HIF-1α interfering siRNA includes a sense strand and an antisense strand:

The sense strand:
(SEQ ID NO: 5)
5′-CUGAUAACGUGAACAAAUATT-3′.
The antisense strand:
(SEQ ID NO: 6)
5′-UAUUUGUUCACGUUAUCAGTT-3′.

The PDNPs are hydrogel-like nanocarriers, formed by intermolecular cross-linking or self-assembly with polypeptide dendrimer as the core skeleton. The PDNPs may be obtained by self-synthesis or commercial purchase.

In some embodiments, the preparation method of the PDNPs-siRNAs may include physical adsorption, chemical cross-linking, or the like.

In some embodiments, in S2, a preparation process of the PDNPs-siRNAs includes: obtaining a mixed interfering solution by dissolving IL-6 interfering siRNA and HIF-1α interfering siRNA using diethyl pyrocarbonate (DEPC)-treated double-distilled water, obtaining a PDNPs solution by dissolving the PDNPs using the DEPC-treated double-distilled water, and obtaining the PDNPs-siRNAs by mixing the mixed interfering solution and the PDNPs solution.

The DEPC is a ribonuclease (RNase) inhibitor. The DEPC may irreversibly inactivate RNase by reacting with histidine residues in proteins and is commonly used to remove the RNase contamination from solutions. The DEPC-treated double-distilled water is ultrapure distilled water that has been treated to inactivate the RNase.

In some embodiments, by using DEPC-treated double-distilled water as a solution, siRNAs can be prevented from being degraded by the RNase, which can help improve the stability of the siRNAs, and the biocompatibility of DEPC is excellent. DEPC is easily decomposed into harmless substances, ensuring no toxic residues when subsequently mixed with vesicles, and ensuring the biosafety of PDNPs.

In some embodiments, the preparation process of the PDNPs includes: in a nitrogen atmosphere, firstly mixing generation 3 poly(epsilon-lysine) (G3-K), benzotriazol-1-yloxytrispyrrolidinophosphonium hexafluorophosphate (PyBOP), 1-hydroxybenzotriazole (HOBT), and O-benzotriazol-N,N,N′,N′-tetramethyluronium hexafluorophosphate (HBTU) in the anhydrous N, N-dimethylformamide (DMF) and stirring at 0° C.; and then, adding N,N-diisopropylethylamine (DIPEA) dropwise to obtain a mixture, stirring the mixture at room temperature, and dialyzing to obtain the polypeptide dendrimer nanoparticles (PDNPs).

The G3-K is a dendritic polymer of epsilon-lysine. The generation 3 denotes that the branching level of poly(epsilon-lysine) is 3. The higher the branching level, the greater the count of branches, and the greater the molecular weight and surface area of the dendritic polymer.

The PyBOP is a carboxyl-activating reagent that promotes amide bond formation. The HOBT is a side reaction inhibitor that ensures directional growth of dendrimer branches while enhancing the efficiency of the condensation reaction. The HBTU is a condensation reagent that ensures the uniform growth of each generation of branches, leading to the formation of a dendritic structure with a uniform particle size. The anhydrous DMF is used as a solvent to provide an anhydrous and homogeneous reaction system. The DIPEA is a pH modifier used to neutralize acidic byproducts of the condensation reaction to produce a dialytically-removable and water-soluble salt.

In some embodiments, deterioration and failure of water-sensitive reagents, such as PyBOP, HOBT, or the like, can be avoided by maintaining a nitrogen atmosphere and using anhydrous DMF, which ensures that the coupling reagent efficiently activates the carboxyl groups of G3-K, to directionally condense with the amine groups of branched amino acids to form amide bonds, constructing highly branched dendritic structures.

In some embodiments, a ratio of the PyBOP, HOBT, HBTU, and anhydrous DMF, the mixing temperature, and the mixing time may be determined according to the actual application scenarios and needs.

In some embodiments, a molar ratio of the G3-K, the PyBOP, the HOBT, and the HBTU is 1:(2-10): 6:6; and a mass-volume ratio of the G3-K to the anhydrous DMF is 1 g: 5 mL.

In some embodiments, the molar ratio of the G3-K, the PyBOP, the HOBT, and the HBTU may also be one of the following: 1:2:6:6, 1:3:6:6, 1:4:6:6, 1:5:6:6, 1:6:6:6, 1:7:6:6, 1:8:6:6, 1:9:6:6, or 1:10:6:6.

In some embodiments, the molar ratio of the G3-K, the PyBOP, the HOBT, and the HBTU may also be one of the following: 1:(2-9): 6:6, 1:(3-10): 6:6, 1:(3-9): 6:6, 1:(2-8): 6:6, 1:(4-10): 6:6, 1:(4-9): 6:6, 1:(2-7): 6:6, 1:(5-10): 6:6, 1:(5-8): 6:6, or 1:(3-8): 6:6.

In some embodiments, the stirring time is 30 minutes at 0° C., and 24 hours at room temperature.

In some embodiments, by setting the molar ratio of the G3-K, the PyBOP, the HOBT, and the HBTU to 1:(2-10): 6:6; and the mass-volume ratio of the G3-K to the anhydrous DMF to 1 g: 5 mL, conditions such as the ratio of the reactants and the temperature are strictly controlled, which helps to ensure the homogeneity of the PDNPs and to minimize batch-to-batch variation.

In some embodiments, a weight ratio of the IL-6 interfering siRNA, HIF-1a interfering siRNA, and PDNPs is 1:1:(16-200); a mixing time for mixing the interfering solution and the PDNPs solution is 30 minutes, and a mixing temperature is room temperature.

In some embodiments, the weight ratio of the IL-6 interfering siRNA, the HIF-1α interfering siRNA, and the PDNPs may also be one of the following: 1:1:16, 1:1:20, 1:1:30, 1:1:40, 1:1:50, 1:1:80, 1:1:100, 1:1:120, 1:1:150, or 1:1:200.

In some embodiments, the weight ratio of the IL-6 interfering siRNA, the HIF-1α interfering siRNA, and the PDNPs may also be one of the following: 1:1:(16-100), 1:1:(20-150), 1:1:(40-160), 1:1:(50-180), 1:1:(30-80), 1:1:(70-160), 1:1:(16-150), 1:1:(120-200), 1:1:(100-200), or 1:1:(16-180).

In some embodiments, by setting the weight ratio of IL-6 interfering siRNA, HIF-1α interfering siRNA, and PDNPs to 1:1:(16-200); the mixing time for mixing the mixed interfering solution and the PDNPs solution to 30 minutes, and the mixing temperature to room temperature, the ratios of the mixed interfering solution and the PDNPs solution may be accurately quantified, ensuring the homogeneity and stability of the PDNPs-siRNAs.

S3, mixing and extruding the vesicles with the PDNPs-siRNAs, to obtain the molecular nanoparticle microcarriers (M@PDNPs-siRNAs).

The molecular nanoparticle microcarrier (M@PDNPs-siRNAs) is a composite delivery system in which PDNPs-siRNAs are encapsulated by vesicles as the outer layer.

In some embodiments, molecular nanoparticle microcarriers (M@PDNPs-siRNAs) of uniform particle size may be prepared by mixing and extruding.

In some embodiments, the mixing and extruding may be performed, but is not limited to, a membrane extruder, a microjet homogenizer, a shear mixer, or the like.

In some embodiments, in S3, the vesicles are mixed with the PDNPs-siRNAs to obtain a mixture, the mixture is placed in a syringe on one side of a membrane extruder, and extruded back and forth several times, to obtain the molecular nanoparticle microcarriers (M@PDNPs-siRNAs).

The membrane extruder is a mechanical mixing device. The membrane extruder includes a filter membrane holder in the middle, the filter membrane holder being mounted with a microporous membrane, and syringes disposed on both sides of the filter membrane holder. During mixing and extruding, a mixture is first placed in one side of the syringe, and the mixture is extruded from the other side of the microporous membrane by pushing a piston of the syringe to provide mechanical pressure to the mixture. When extruded, the mixture is forced to break up and co-mingle as it passes through the microporous membrane, completing the structural reorganization. Several back and forth extrusions causes the vesicles to wrap around the PDNPs-siRNAs, and the M@PDNPs-siRNAs may be obtained.

In some embodiments, the particle size of the M@PDNPs-siRNAs may be adjusted by using microporous membranes with different pore sizes.

In some embodiments, by the membrane extruder, the M@PDNPs-siRNAs with a uniform particle size can be prepared without the need of an expensive mixing device, which helps to reduce production costs while safeguarding product quality.

In some embodiments, the count of extrusions, and the weight ratio of the vesicles to the PDNPs-siRNAs may be determined according to the actual application scenarios and needs. For example, the weight ratio of the vesicles to the PDNPs-siRNAs is 1:1.

In some embodiments, when the weight ratio of the vesicles is too low, the vesicles are unable to sufficiently encapsulate the PDNPs, resulting in a low yield of the M@PDNPs-siRNAs; whereas when the weight ratio of the vesicles is too high, the PDNPs wrapped by the vesicles are too few, resulting in too low delivery efficiency of M@PDNPs-siRNAs. By setting the weight ratio of the vesicles to the PDNPs-siRNAs to 1:1, the component ratio of the M@PDNPs-siRNAs can be precisely controlled to balance the yield and delivery efficiency of M@PDNPs-siRNAs.

In some embodiments, the present disclosure applies MSCs vesicles expressing CXCR4 and PSGL-1, which can enrich M@PDNPs-siRNAs at the inflammation sites, exhibiting good inflammation targeting property; and the present disclosure designs the PDNPs-siRNAs, the PDNPs is positively charged and can adsorb negatively charged nucleic acids or drugs, which realizes prolonged drug administration and sustained drug release, with strong practicality.

In some embodiments, a therapeutically effective amount of a medication is administered to a patient suffering from the RA, and the medication includes the M@PDNPs-siRNAs as described above.

The present disclosure prepares M@PDNPs-siRNAs that may be used for cellular transfection and intravenous administration of a drug, which allows for safe and targeted drug delivery.

The following is examples of the preparation of a molecular nanoparticle microcarrier and experimental validation, providing a further description of the molecular nanoparticle microcarrier and the related method provided by some embodiments of the present disclosure.

### Example 1: Preparation of the MSCs Vesicles Overexpressing CXCR4 and PSGL-1

### (I) Preparation of the MSCs Overexpressing CXCR4 and PSGL-1

### (a) Construction of the Recombinant Vector for the MSCs Overexpressing CXCR4 and PSGL-1

The recombinant vectors overexpressing CXCR4 and PSGL-1, respectively, were constructed using the molecular biology techniques, and the used vector was the plv-IRES, with the insertion of the target sequences (CXCR4 or PSGL-1), three repetitive FLAG tag sequences, the EGFP sequence, and the puromycin resistance sequence in the open reading frame.

### (b) Lentiviral Packaging

The lentiviral packaging system was used to package the lentivirus in 293T cells. The lentiviral packaging system includes two separate plasmid mixtures: a plasmid mixture for packaging CXCR4 lentivirus and a plasmid mixture for packaging PSGL-1 lentivirus. The plasmid mixture for packaging CXCR4 lentivirus includes: the plv-IRES-CXCR4 (20 μg), the psPAX2 (15 μg), and the PMD2G (5 μg). The plasmid mixture for packaging PSGL-1 lentivirus includes: the plv-IRES-PSGL-1 (20 μg), the psPAX2 (15 μg), and the PMD2G (5 μg).

Firstly, the 293T cells were cultured to 70% confluency, and after the fluid exchange, the plasmid mixture for packaging CXCR4 lentivirus and p3000 were mixed and left to stand at room temperature for 15 minutes, and then added to the Lip3000 transfection reagent. The resulting suspension was added drop by drop to 293T cells and the supernatant was collected after 48 hours of incubation, to obtain the CXCR4 lentiviral suspension. The plasmid mixture for packaging PSGL-1 lentivirus and p3000 were mixed and left to stand at room temperature for 15 minutes, and then added to the Lip3000 transfection reagent. The resulting suspension was added drop by drop to 293T cells and the supernatant was collected after 48 hours of incubation, to obtain the PSGL-1 lentiviral suspension.

### (c) Lentiviral Infection of the MSCs

The MSCs were cultured in a 6-well plate to 70% confluency, 1 mL of the CXCR4 lentiviral suspension was added, the co-transfection reagent was added, and the cells were cultured for 48 hours before puromycin was added to perform the antibiotic screening. After removing the dead cells, the remaining cells were CXCR4-overexpressing cells. Subsequently, the PSGL-1 lentiviral suspension was added to the CXCR4-overexpressing cells, the co-transfection reagent was added, and the cells were cultured for 48 hours before puromycin was added to perform the antibiotic screening again. After removing the dead cells, the remaining cells were MSCs overexpressing CXCR4 and PSGL-1.

### (II) Preparation of Vesicles

The MSCs overexpressing CXCR4 and PSGL-1 were cultivated in complete medium, and when the cell density reached about 80%, the complete medium was changed to serum-free medium, the cytochalasin B was added, and the cells were treated with the cytochalasin B at a working concentration of 10 μg/mL for 30 minutes.

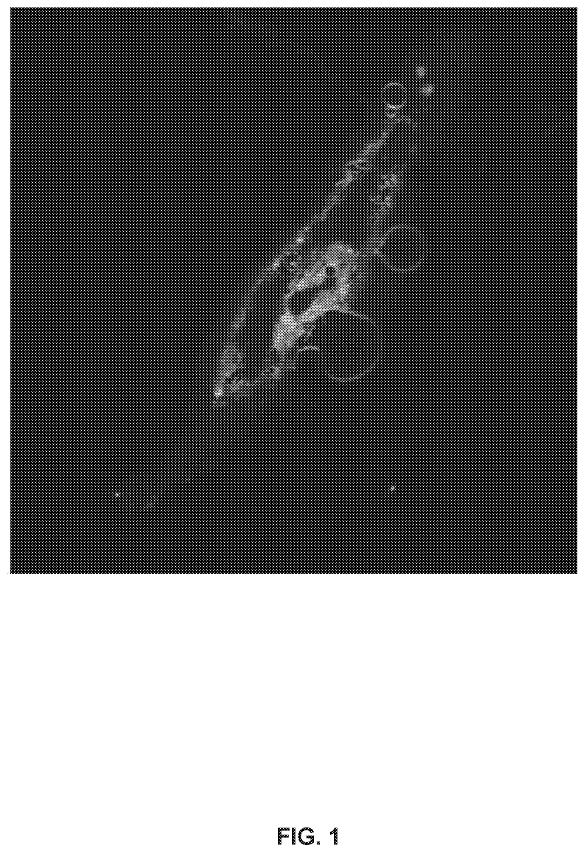

FIG. 1 is a schematic diagram of MSCs vesicles overexpressing CXCR4 and PSGL-1 according to Example 1 of the present disclosure. As shown in FIG. 1, after the treatment of the cytochalasin B, obvious vesicles are observed in the MSCs.

The treated cells were digested with trypsin, centrifuged at 1000 rpm at room temperature for 5 minutes, washed once with the PBS, and centrifuged again at 1000 rpm at room temperature for 5 minutes. The supernatant was taken, centrifuged at 6000 rpm at room temperature for 15 minutes, and the obtained precipitate was the vesicles.

### Example 2: Preparation of the PDNPs-siRNAs

### (I) Preparation of the PDNPs

The G3-K (1.00 g, 0.12 mmol), the PyBOP (0.12 g/0.6 mmol), the HOBT (97 mg, 0.72 mmol), and the HBTU (273 mg, 0.72 mmol) were first mixed in 5 mL of the anhydrous DMF in a nitrogen atmosphere and stirred at 0° C. for 30 minutes, then, several drops of the DIPEA were added. The mixture was further stirred at room temperature for 24 hours. The PDNPs were purified by dialysis against DMF and distilled water for 3 days.

### (II) Preparation of the PDNPs-siRNAs

The IL-6 interfering siRNA and HIF-1α interfering siRNA were dissolved using DEPC-treated double-distilled water, and 125 ng of each siRNA was taken and mixed well to 250 ng.

The PDNPs were dried and dissolved in DEPC-treated double-distilled water. The different masses of the PDNPs were taken as 0, 0.5, 1, 2, 5, 10 and 20 μg.

The two solutions were mixed thoroughly, and reacted at room temperature for 30 min, then the AGE analysis was performed.

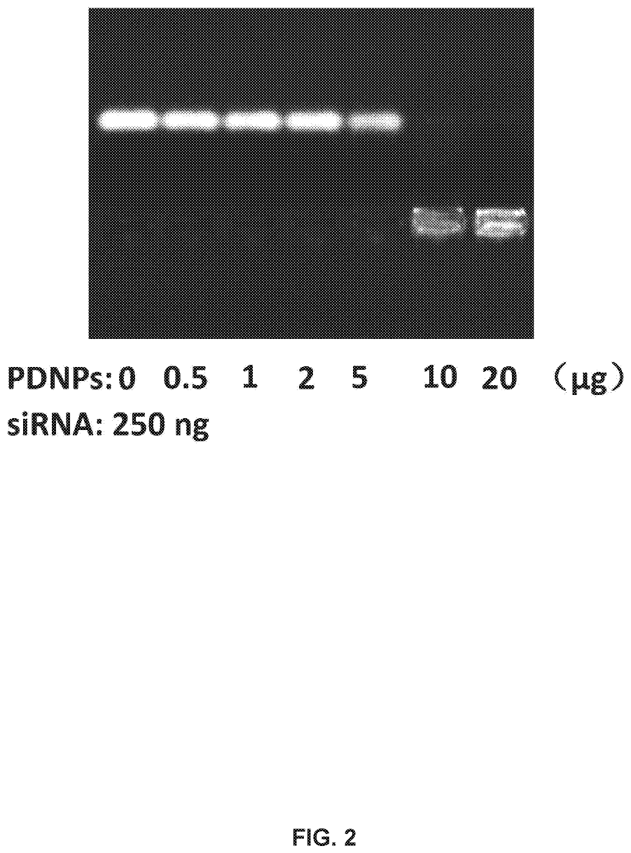

FIG. 2 is an agarose gel electrophoresis (AGE) image of small interfering ribonucleic acids-loaded polypeptide dendrimer nanoparticles (PDNPs-siRNAs) according to Example 2 of the present disclosure. As shown in FIG. 2, when the mass of siRNAs is 250 ng and the mass of PDNPs is 10 μg, the siRNAs may be completely adsorbed to obtain the PDNPs-siRNAs.

In this embodiment, the IL-6 interfering siRNA and the HIF-1α interfering siRNA were synthesized by the company.

The IL-6 interfering siRNA includes a sense strand and an antisense strand.

The sense strand:
(SEQ ID NO: 3)
5′-CCUCUGGUCU UCUGGAGUATT-3′.
The antisense strand:
(SEQ ID NO: 4)
5′-UACUCCAGAAGACCAGAGGTT-3′.

The HIF-1α interfering siRNA includes a sense strand and an antisense strand.

The sense strand:
(SEQ ID NO: 5)
5′-CUGAUAACGUGAACAAAUATT-3′.
The antisense strand:
(SEQ ID NO: 6)
5′-UAUUUGUUCACGUUAUCAGTT-3′.

### Example 3: Preparation of the M@PDNPs-siRNAs

The vesicles obtained in Example 1 were collected and the protein concentration was determined. The M@PDNPs-siRNAs were prepared by uniformly mixing the vesicles (from Example 1) and the PDNPs-siRNAs (from Example 2) at equal mass. The mixture was added to the syringe on one side of the membrane extruder, and then extruded back and forth the syringe 10 times, to obtain the M@PDNPs-siRNAs.

The prepared M@PDNPs-siRNAs were subjected to TEM analysis.

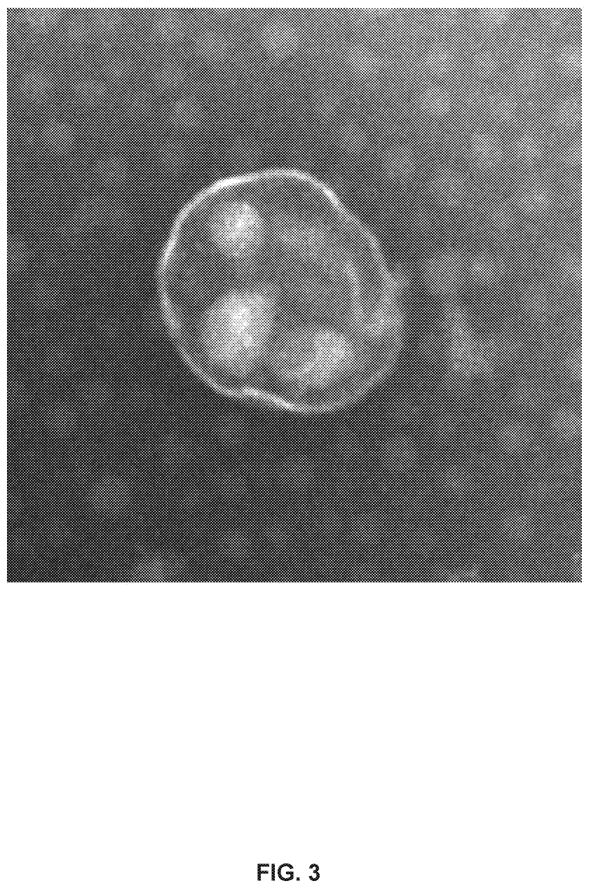

FIG. 3 is a transmission electron microscope (TEM) image of a molecular nanoparticle microcarrier (M@PDNPs-siRNAs) according to Example 3 of the present disclosure. As shown in FIG. 3, the cell membrane structure is clearly visible at the edge of M@PDNPs-siRNAs.

### Example 4: Experimental Procedure for Detecting Transfection Efficiency of M@PDNPs-siRNAs Transfected into the Synovial Fibroblasts

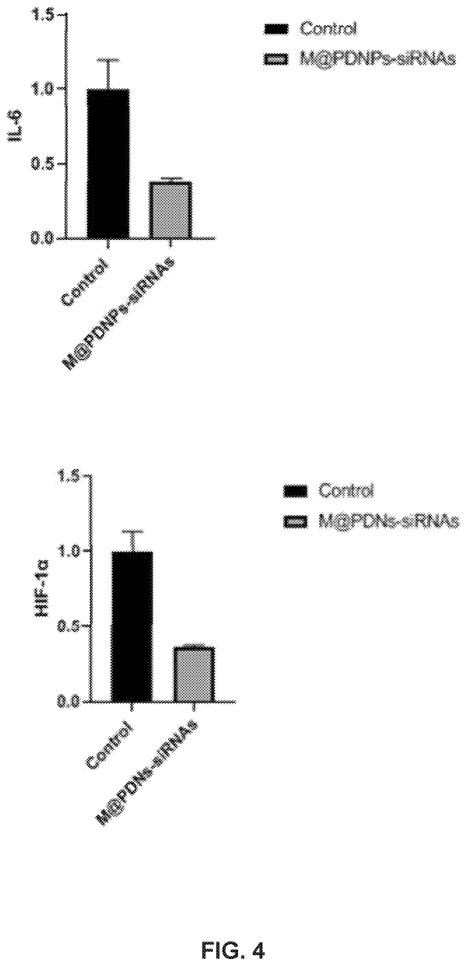

The synovial fibroblasts were cultured with the complete medium. When the cells expanded to a density of about 60%, the M@PDNPs-siRNAs were added. After a total of 48 hours of co-culture, the cells were collected, and the expression of the target genes was detected by the real-time quantitative polymerase chain reaction (PCR) with reference to FIG. 4.

FIG. 4 is a chart showing the effect of inhibiting target gene expression after transfection of synovial fibroblasts with M@PDNPs-siRNAs according to some embodiments of the present disclosure.

As shown in FIG. 4, after the treating synovial fibroblasts with the M@PDNPs-siRNAs, the expression of IL-6 and HIF-1α is significantly reduced.

### Example 5: Targeting Validation Test of the M@PDNPs-siRNAs

Using a collagen-induced RA mouse model, the M@PDNPs-siRNAs (siRNAs carrying a cy5.5 tag) were injected via the tail vein, and the aggregation of the M@PDNPs-siRNAs at the inflammatory sites was observed using small animal imaging 24 hours later.

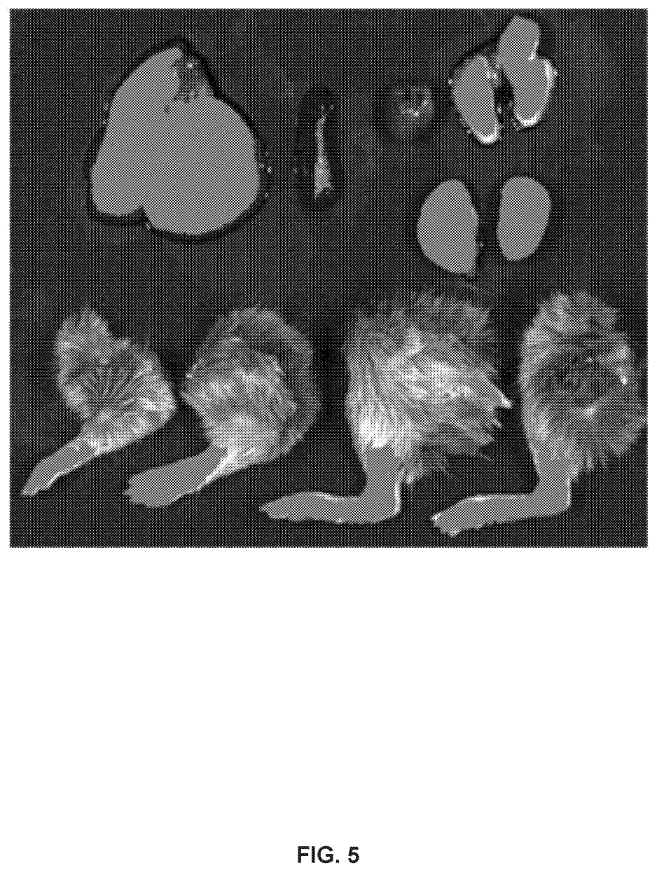

FIG. 5 is an image showing the effect of targeting validation of M@PDNPs-siRNAs according to some embodiments of the present disclosure.

As shown in FIG. 5, the M@PDNPs-siRNAs are enriched at the inflammatory joints, demonstrating that the M@PDNPs-siRNAs are well targeted.

It should be noted that the above descriptions are merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

What is claimed is:

1. A method for preparing molecular nanoparticle microcarriers, comprising:
S1, stimulating mesenchymal stem cells (MSCs) to produce vesicles and collecting the vesicles by centrifugation;
wherein the MSCs overexpress CXCR4 and PSGL-1, and a preparation process of the MSCs includes:
(a) constructing a recombinant vector of CXCR4 and PSGL-1 using molecular biology techniques;
(b) packaging lentivirus in 293T cells using the recombinant vector of CXCR4 and PSGL-1; and
(c) collecting the lentivirus, infecting the MSCs with concentrated lentivirus, and screening positive cells expressing both CXCR4 and PSGL-1 using antibiotic tags and a flow cytometry to obtain a stable MSCs line that overexpresses CXCR4 and PSGL-1;
S2, preparing small interfering ribonucleic acids-loaded polypeptide dendrimer nanoparticles (PDNPs-siRNAs); and
S3, mixing and extruding the vesicles with the PDNPs-siRNAs, to obtain the molecular nanoparticle microcarriers (M@PDNPs-siRNAs).

2. The method according to claim 1, wherein in S1, a preparation process of the vesicles includes:
treating the MSCs overexpressing CXCR4 and PSGL-1 with cytochalasin B, and obtaining the vesicles of the MSCs overexpressing CXCR4 and PSGL-1 by ultra-high speed centrifugation.

3. The method according to claim 1, wherein in S2, a preparation process of the PDNPs-siRNAs includes:
obtaining a mixed interfering solution by dissolving IL-6 interfering siRNA and HIF-1α interfering siRNA using diethyl pyrocarbonate (DEPC)-treated double-distilled water;
obtaining a PDNPs solution by dissolving the PDNPs using the DEPC-treated double-distilled water; and
obtaining the PDNPs-siRNAs by mixing the mixed interfering solution and the PDNPs solution.

4. The method according to claim 3, wherein
a weight ratio of the IL-6 interfering siRNA, the HIF-1α interfering siRNA, and the PDNPs is 1:1:(16-200); a mixing time of the mixed interfering solution and the PDNPs solution is 30 minutes, and a mixing temperature of the mixed interfering solution and the PDNPs solution is room temperature.

5. The method according to claim 3, wherein
a preparation process of the PDNPs includes:
in a nitrogen atmosphere, firstly mixing generation 3 poly(epsilon-lysine) (G3-K), benzotriazol-1-yloxytrispyrrolidinophosphonium hexafluorophosphate (PyBOP), 1-hydroxybenzotriazole (HOBT), and O-benzotriazol-N,N,N′,N′-tetramethyluronium hexafluorophosphate (HBTU) in anhydrous N,N-dimethylformamide (DMF) and stirring at 0° C.; and
then, adding N,N-diisopropylethylamine (DIPEA) dropwise to obtain a mixture, stirring the mixture at room temperature, and dialyzing to obtain the polypeptide dendrimer nanoparticles (PDNPs).

6. The method according to claim 5, wherein
a molar ratio of the G3-K, the PyBOP, the HOBT, and the HBTU is 1:(2-10): 6:6;
a mass-volume ratio of the G3-K to the anhydrous DMF is 1 g: 5 mL; and
a stirring time is 30 minutes at 0° C. and a stirring time is 24 h at room temperature.

7. The method according to claim 1, wherein
in S3, the vesicles are mixed with the PDNPs-siRNAs to obtain a mixture, the mixture is placed in a syringe on one side of a membrane extruder, and extruded back and forth several times, to obtain the molecular nanoparticle microcarriers (M@PDNPs-siRNAs).

8. The method according to claim 7, wherein
a mass ratio of the vesicles to the PDNPs-siRNAs is 1:1.


SEQUENCE LISTING (6 sequences)

SEQ ID NO: 1 - DNA, 1071 bp, synthetic construct
  source 1..1071: mol_type = other DNA
atgtccattcctttgcctcttttgcagatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaa

SEQ ID NO: 2 - DNA, 1287 bp, synthetic construct
  source 1..1287: mol_type = other DNA
atggcagtgggggccagtggtctagaaggagataagatggctggtgccatgcctctgcaactcctcctgttgctgatcctactgggccctggcaacagcttgcagctgtgggacacctgggcagatgaagccgagaaagccttgggtcccctgcttgcccgggaccggagacaggccaccgaatatgagtacctagattatgatttcctgccagaaacggagcctccagaaatgctgaggaacagcactgacaccactcctctgactgggcctggaacccctgagtctaccactgtggagcctgctgcaaggcgttctactggcctggatgcaggaggggcagtcacagagctgaccacggagctggccaacatggggaacctgtccacggattcagcagctatggagatacagaccactcaaccagcagccacggaggcacagaccactcaaccagtgcccacggaggcacagaccactccactggcagccacagaggcacagacaactcgactgacggccacggaggcacagaccactccactggcagccacagaggcacagaccactccaccagcagccacggaagcacagaccactcaacccacaggcctggaggcacagaccactgcaccagcagccatggaggcacagaccactgcaccagcagccatggaagcacagaccactccaccagcagccatggaggcacagaccactcaaaccacagccatggaggcacagaccactgcaccagaagccacggaggcacagaccactcaacccacagccacggaggcacagaccactccactggcagccatggaggccctgtccacagaacccagtgccacagaggccctgtccatggaacctactaccaaaagaggtctgttcatacccttttctgtgtcctctgttactcacaagggcattcccatggcagccagcaatttgtccgtcaactacccagtgggggccccagaccacatctctgtgaagcagtgcctgctggccatcctaatcttggcgctggtggccactatcttcttcgtgtgcactgtggtgctggcggtccgcctctcccgcaagggccacatgtaccccgtgcgtaattactcccccaccgagatggtctgcatctcatccctgttgcctgatgggggtgaggggccctctgccacagccaatgggggcctgtccaaggccaagagcccgggcctgacgccagagcccagggaggaccgtgagggggatgacctcaccctgcacagcttcctcccttag

SEQ ID NO: 3 - RNA, 21 nt, synthetic construct
  source 1..21: mol_type = other RNA
cctctggtcttctggagtatt

SEQ ID NO: 4 - RNA, 21 nt, synthetic construct
  source 1..21: mol_type = other RNA
tactccagaagaccagaggtt

SEQ ID NO: 5 - RNA, 21 nt, synthetic construct
  source 1..21: mol_type = other RNA
ctgataacgtgaacaaatatt

SEQ ID NO: 6 - RNA, 21 nt, synthetic construct
  source 1..21: mol_type = other RNA
tatttgttcacgttatcagtt